(19) United States
(12) Patent Application Publication
(10) Pub. No.: US 2013/0309171 A1
(43) Pub. Date: Nov. 21, 2013

(54) PRE-TARGETED NANOPARTICLE SYSTEM AND METHOD FOR LABELING BIOLOGICAL PARTICLES

(71) Applicant: University of Washington through its Center for Commercialization, Seattle, WA (US)

(72) Inventors: Miqin Zhang, Bothell, WA (US); Oliver W. Press, Seattle, WA (US); Jonathan Whitney Gunn, Seattle, WA (US); Steven I. Park, Mountlake Terrace, WA (US)

(73) Assignee: University of Washington through its Center for Commercialization, Seattle, WA (US)

(21) Appl. No.: 13/872,827

(22) Filed: Apr. 29, 2013

Related U.S. Application Data
(63) Continuation of application No. PCT/US2011/058447, filed on Oct. 28, 2011.
(60) Provisional application No. 61/408,442, filed on Oct. 29, 2010.

(51) Int. Cl. A61K 47/48; A61K 48/00; A61K 49/00

(52) U.S. Cl. CPC A61K 47/48723; A61K 49/0004; A61K 48/0041
USPC 424/9.1; 424/178.1; 435/7.1

(57) ABSTRACT

Nanoparticle system and method for labeling, detecting, and treating biological particles. In the method, targeting functionality (fusion protein) and therapeutic/imaging modalities (nanoparticle) are separated.

### CROSS-REFERENCE TO RELATED APPLICATION

This application is a continuation of International Application No. PCT/US2011/058447, filed Oct. 28, 2011, which claims the benefit of U.S. Patent Application No. 61/408,442, filed Oct. 29, 2010, each expressly incorporated herein by reference in its entirety.

### STATEMENT OF GOVERNMENT LICENSE RIGHTS

This invention was made with Government support under 5R01CA076287, R01CA134213, R01CA119408, and R01EB006043 awarded by National Institutes of Health (NIH). The Government has certain rights in the invention.

### BACKGROUND OF THE INVENTION

Nanoparticle-based targeting agents, such as liposomes, quantum dots, and superparamagnetic particles, have been developed that exhibit excellent biomedical imaging and therapeutic capacities. These nanoparticles capitalize on the specificity of targeting ligands attached at their surfaces, such as antibodies, peptides, and small molecules, to bind targeted cells, thus providing highly selective cell labeling. Nanoparticles modified with these targeting ligands are able to identify cancerous lesions, angiogenesis, and age-related macular degeneration.

With a robust pipeline of biomolecular ligands being developed against different diseases, more and more new molecular-targeted nanoparticles are anticipated. However, each ligand is commonly specific to only one or a limited few target receptors, necessitating development of multiple nanoparticle formulations with different targeting ligands for different diseases. The development of each nanoparticle formulation requires extensive, costly design and experimentation to resolve a series of common issues, such as possible biomolecule deactivation, low ligand:nanoparticle attachment, and colloidal stability. In addition, the use of large targeting molecules, such as antibodies, can potentially increase nanoparticle immunogenicity, shorten its blood half-life, and limit its vascular extravasation in vivo.

Fusion proteins, such as 1F5 (anti-CD20), Lym-1 (anti-HLA-DR), and CC49 (anti-TAG-72), have demonstrated success in binding a range of different cancer types, including B-cell lymphoma, gastrointestinal cancers, and leukemia. These fusion proteins are used in combination with biotinylated radioisotopes (¹¹¹In, ⁹⁰Y) in radioimmunotherapy to target cancer cells while at the same time limiting radiation exposure to healthy tissue. This targeting strategy has been particularly successful in treating lymphomas.

Despite the advances in nanoparticle systems for targeting biological targets noted above, a need exists for a universal nanoparticle system that can accommodate different targeting ligands without alternating pharmacokinetic profile of the nanoparticle itself, thereby decreasing the time and cost associated with nanoparticle targeting of biological targets. The present invention seeks to fulfill this need and provides further related advantages.

### SUMMARY OF THE INVENTION

The present invention provides a nanoparticle system and method based on a pre-treatment strategy capable of binding diverse cell targets. In the method, targeting functionality (fusion protein) and therapeutic/imaging modalities (nanoparticle) are separated.

In one aspect, the invention provides a method for labeling a biological particle. In one embodiment, the method includes contacting a fusion protein-labeled biological particle having an affinity to a binding partner with a nanoparticle comprising the binding partner to provide a nanoparticle-labeled biological particle.

In another embodiment, the method includes:

(a) contacting a biological particle with a fusion protein having a binding region having an affinity to a binding partner and a targeting region having an affinity toward the biological particle to provide a fusion protein-labeled biological particle; and

(b) contacting the fusion protein-labeled biological particle with a nanoparticle comprising the binding partner to provide a nanoparticle-labeled biological particle.

Biological particles that may be effectively labeled in accordance with the invention include cells, bacteria, viruses or viral particles, and other biomarkers. Representative cells include stem cells, blood cells, tissue cells, and abnormal cells, such as cancer cells.

In one embodiment, the nanoparticle is a magnetic nanoparticle. In another embodiment, the nanoparticle further includes a therapeutic agent.

In other aspects, the invention provides methods for labeling and treating cancer cells. In one embodiment, the method for labeling cancer cells includes:

(a) contacting a cancer cell with a fusion protein having a biotin-binding region and a targeting region having an affinity toward the cancer cell to provide a fusion protein-labeled cancer cell; and

(b) contacting the fusion protein-labeled cancer cell with a biotinylated nanoparticle to provide a nanoparticle-labeled biological particle.

The invention also provides a method for treating cancer by delivering a chemotherapeutic agent to a cancer cell. In one embodiment, the method for treating cancer cells includes contacting a fusion protein-labeled cancer cell having an affinity to a binding partner with a nanoparticle comprising the binding partner and a chemotherapeutic agent.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIGS. 1A-1D illustrate the physiochemical properties of a representative nanoparticle of the invention. FIG. 1A compares Fourier transform infrared (FTIR) of uncoated nanoparticles, chitosan-PEG-coated nanoparticles (NP), and biotinylated-NP binding SA (NPB-SA). FIG. 1B compares fluorescence analysis showing that biotinylated nanoparticles (NPB-AF647) enhanced binding to the avidin of the biotective agent, compared with control nanoparticles (i.e., NP and NP-AF647). FIG. 1C compares hydrodynamic sizes of nanoparticles with and without fluorophores or biotin (i.e., NP, NP-AF647, and NPB-AF647). FIG. 1D compares zeta potentials of nanoparticles with and without biotin (i.e., NP, NP-AF647, and NPB-AF647).

FIGS. 2A and 2B illustrate cell targeting with NPB-AF647, a representative nanoparticle of the invention. FIG. 2A compares flow cytometry analysis of Ramos and Jurkat cells treated with NPB-AF647 after pretreatment with different fusion proteins (FPs) or receiving no FP pretreatment. Ramos and Jurkat cells receiving neither FP nor NP treatment are given as references (right column). FIG. 2A illustrates dependence of NPB-AF647 cell-binding on FP pre-labeling demonstrated by flow cytometry where the secondary antibody, goat-antimouse-Cy3 (GAM-Cy3), indicates FP labeling.

FIG. 3 compares the interaction of NPB-AF647, a representative nanoparticle of the invention, with cells. Nanoparticle-Ramos cell interactions analyzed by Differential Interference Contrast (DIC, column 1) and confocal fluorescence microscopy (columns 2-5). Row 1, Ramos cells pretreated with anti-CD20 FP and exposed to NPB-AF647; row 2, Ramos cells pretreated with anti-CD20 FP and exposed to NP-AF674 (control particles); row 3, Ramos cells with no pretreatment (control cells) exposed to NPB-AF674. The nanoparticles were labeled red (AF674), anti-CD20 on cells labeled yellow (GAM-Cy3), cell membranes labeled green (WGA-594), and cell nuclei labeled blue (DAPI). NPB showed no cell binding to Ramos cells receiving no FP pretreatment (row 3).

FIGS. 4A-4C illustrate internalization of NPB-AF647, a representative nanoparticle of the invention, by target cells. FIG. 4A is a transmission electron microscopy (TEM) image of Ramos cells treated with the anti-CD20 FP and exposed to NPB-AF647 showing a large population of nanoparticles (black dots) inside cellular endosomes (circular white areas with black dots). The white square frame (top image) marks the region of interest in an endosome shown in the higher magnification micrograph (bottom image) showing the dispersed nanoparticles in the endosome. FIG. 4A is a TEM image at high magnification showing the characteristic lattice planes of the nanoparticle crystal (circled areas) taken from a cellular endosome (marked by the arrow in the inset) containing nanoparticles. The scale bar is 2 nm in the image and 0.5 μm in the inset. FIG. 4C is a TEM micrograph of Ramos cells receiving no anti-CD20 FP pretreatment, showing no particle internalization after exposure to NPB-AF647.

### DETAILED DESCRIPTION OF THE INVENTION

The present invention provides a nanoparticle system and method based on a pre-treatment strategy capable of binding diverse cell targets. In the method, targeting functionality (fusion protein) and therapeutic/imaging modalities (nanoparticle) are separated. The method avoids the complications associated with direct conjugation of targeting molecules to nanoparticles and can be used with any one of many available fusion protein targeting constructs without the need of developing a new nanoparticle system for each disease.

In one aspect, the invention provides a method for labeling a biological particle. In one embodiment, the method includes:

(a) contacting a biological particle with a fusion protein having a binding region having an affinity to a binding partner and a targeting region having an affinity toward the biological particle to provide a fusion protein-labeled biological particle; and

(b) contacting the fusion protein-labeled biological particle with a nanoparticle comprising the binding partner to provide a nanoparticle-labeled biological particle.

The method of the invention is effective to label and therefore detect select biological particles. As used herein, the term “biological particle” refers to a particle that occurs in nature having one or more surface features (e.g., receptors, epitopes) that allows the particle to be targeted by a suitable fusion protein. As used herein, the term “fusion protein” refers to a protein having at least two domains: a first domain that includes a targeting region that has an affinity toward the biological particle (e.g., surface feature) and a second domain that includes a binding region having an affinity to a binding partner.

In the method of the invention, the biological particle is pre-targeted with the fusion protein. As used herein, the term “pre-targeted” refers to the initial labeling of the biological particle with a suitable fusion protein to provide a biological particle to which is bound the suitable fusion protein (i.e., fusion protein-labeled biological particle). The association of the fusion protein to the biological particle is through the affinity of the targeting region of the fusion protein to the corresponding surface feature of the biological particle. Because of the dual nature of the fusion protein, the fusion protein imparts a binding region to the pre-targeted biological particle.

In accordance with the method of the invention, once the biological particle is pre-targeted, the biological particle is then targeted and labeled by a nanoparticle that includes a binding partner having an affinity to the pre-targeted biological particle. Labeling of the pre-targeted biological particle by the nanoparticle is through the affinity of the binding region of the fusion protein to the binding partner of the nanoparticle. By this method, the biological particle is pre-targeted by the fusion protein and then ultimately targeted and labeled by the nanoparticle.

Thus, in one aspect, the invention provides a method for labeling a biological particle that includes contacting a fusion protein-labeled biological particle having an affinity to a binding partner with a nanoparticle comprising the binding partner to provide a nanoparticle-labeled biological particle.

Biological particles that may be effectively labeled in accordance with the invention include cells, bacteria, viruses or viral particles, and other biomarkers. Representative cells include stem cells (e.g., embryonic and adult stem cells), blood cells (e.g., lymphocytes, monocytes, neutrophils, eosinophils, basophils, red blood cells, and platelets) and tissue cells (e.g., connective tissue cells, epithelial cells, muscle cells, or nerve cells). In certain embodiments, the cell is an abnormal cell, such as cancer cell (e.g., a metastatic cancer cell).

The fusion protein useful in the method of the invention includes a targeting region having an affinity toward the biological particle. In one embodiment, the targeting region comprises a single chain antibody or functional fragment thereof.

The fusion protein useful in the method of the invention also includes a binding region having an affinity toward a binding partner. In the method of the invention, the nanoparticle includes the binding partner. The fusion protein's binding region having an affinity toward a binding partner and the nanoparticle's binding partner make up a binding pair.

The fusion protein binding regions are proteinaceous binding regions effective for binding their complements (e.g. receptors/ligands). Suitable binding regions include proteinaceous regions that bind mono-, di-, and polysaccharides (e.g., carbohydrates), nucleic acids and their fragments (e.g., DNA and RNA aptamers), and small molecules. In one embodiment, binding regions include antibody or antibody fragments effective for binding their haptens. Other suitable proteinaceous binding regions include antibody binding protein A, chitin binding protein, maltose binding protein, and histidine-Ni or -Co.

Representative fusion protein binding regions include avidin, streptavidin, neutravidin, or functional fragments thereof. For these embodiments, the nanoparticle's binding partner is a biotin or analog or derivative thereof (e.g., desthiobiotin).

The biological particle labeling is effected by contacting the fusion protein-labeled biological particle with a nanoparticle including the binding partner to provide a nanoparticle-labeled biological particle.

The particle useful in the method of invention has nanoscale dimensions. Suitable particles have a physical size less than about 50 nm. In certain embodiments, the nanoparticles have a physical size from about 2 to about 30 nm. As used herein, the term “physical size” refers to the overall diameter of the nanoparticle, including core (as determined by TEM) and coating thickness. Suitable particles have a mean core size of from about 2 to about 25 nm. In certain embodiments, the nanoparticles have a mean core size of about 7 nm. As used herein, the term “mean core size” refers to the core size determined by TEM. Suitable particles have a hydrodynamic size less than about 250 nm. In certain embodiments, the nanoparticles have a hydrodynamic size from about 10 to about 250 nm. In certain embodiments, the nanoparticles have a hydrodynamic size of about 33 nm. As used herein, the term “hydrodynamic size” refers to the radius of a hard sphere that diffuses at the same rate as the particle under examination as measured by DLS. The hydrodynamic radius is calculated using the particle diffusion coefficient and the Stokes-Einstein equation given below, where k is the Boltzmann constant, T is the temperature, and η is the dispersant viscosity:

$${R}_{H} = \frac{kT}{6 π η D} .$$

A single exponential or Cumulant fit of the correlation curve is the fitting procedure recommended by the International Standards Organization (ISO). The hydrodynamic size extracted using this method is an intensity weighted average called the Z average.

In certain embodiments, the nanoparticle includes one or more imaging agents. Suitable imaging agents include magnetic resonance imaging agents, fluorescent agents, ultrasound imaging agents, radiolabels, surface plasmon resonance imaging agents. In certain embodiments, the nanoparticle has a core that renders the nanoparticle itself suitable as a magnetic resonance imaging agent, ultrasound imaging agent, radiolabel, or surface plasmon resonance imaging agent.

Suitable magnetic resonance imaging agents include iron- (e.g., iron oxide) and gadolinium-based agents.

Suitable fluorescent agents include fluorescent agents that emit visible and near-infrared light (e.g., fluorescein and cyanine derivatives). Representative fluorescent agents include fluorescein, OREGON GREEN 488, ALEXA FLUOR 555, ALEXA FLUOR 647, ALEXA FLUOR 680, Cy5, Cy5.5, and Cy7.

Suitable ultrasound imaging agents include carbon- and metal-based agents.

Suitable radiolabels includes ¹³¹I for radioimaging and ⁶⁴Cu, ¹⁸F, and ¹¹C for positron emission tomography (PET).

Suitable surface plasmon resonance imaging agents include gold-based agents.

In certain embodiments, the nanoparticle includes one or more therapeutic agents. Suitable therapeutic agents include conventional therapeutic agents, such as small molecules; biotherapeutic agents, such as peptides, proteins, and nucleic acids (e.g., DNA, RNA, cDNA, siRNA); and cytotoxic agents, such as alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones, and anti-androgens.

In certain embodiments, the therapeutic agent is a chemotherapeutic agent, a gene therapeutic agent, or a therapeutic radioisotope.

In certain embodiments, the gene therapeutic agent is a thymidine kinase gene or a p53 gene, or an siRNA is against Cyclin D1, Cyclin D2, or STAT3.

In certain embodiments, the therapeutic radioisotope is ¹¹¹In ⁹⁰Y, or ¹³¹I.

Representative cytotoxic agents include BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminoglutethimide, anastrozole, amsacrine, asparaginase, mitoxantrone, mitotane, and amifostine.

In certain embodiments, the nanoparticle includes one or more imaging agents and one or more therapeutic agents.

In one embodiment, the nanoparticle is a magnetic nanoparticle. In these embodiments, the nanoparticle includes a core material. For magnetic resonance imaging applications, the core material is a material having magnetic resonance imaging activity (e.g., the material is paramagnetic). In certain embodiments, the core material is a magnetic material. In other embodiments, the core material is a semiconductor material. Representative core materials include ferrous oxide, ferric oxide, silicon oxide, polycrystalline silicon oxide, silicon nitride, aluminum oxide, germanium oxide, zinc selenide, tin dioxide, titanium, titanium dioxide, nickel titanium, indium tin oxide, gadolinium oxide, stainless steel, gold, and mixtures thereof.

In certain embodiments, the magnetic nanoparticle further includes an imaging agent. In certain embodiments, the magnetic nanoparticle further includes a therapeutic agent. In certain embodiments, the magnetic nanoparticle further includes an imaging agent and a therapeutic agent. Suitable imaging and therapeutic agents include those noted above.

The therapeutic agent can be covalently coupled to the nanoparticle (e.g., copolymer coating) or non-covalently (e.g., ionic) associated with the nanoparticle. For therapeutic agent delivery, the therapeutic agent can be covalently linked (e.g., through a cleavable linkage), physically adsorbed to (e.g., electrostatic or van der Waals interactions), or embedded within the nanoparticle (or copolymer coating).

In one embodiment, the nanoparticle includes a core having a surface with a copolymer forming a coating on the surface. The copolymer is anchored to the core surface (e.g., oxide surface) by interactions between the core surface and the amine and hydroxyl groups on the copolymer's chitosan backbone. It is believed that the coating is a multi-layered mesh that encapsulates the core.

The coating of the nanoparticle is formed from a copolymer comprising a chitosan and a poly(ethylene oxide) oligomer. In one embodiment, the copolymer is a graft copolymer having a chitosan backbone and pendant poly(ethylene oxide) oligomer side chains.

Chitosan is a linear polysaccharide composed of randomly distributed β-(1-4)-linked D-glucosamine (deacetylated unit) and N-acetyl-D-glucosamine (acetylated unit). Suitable chitosans useful in making the copolymers useful in the invention have a molecular weight (weight average, Mw) of from about 0.3 to about 50 kDa. In certain embodiments, the chitosan has a molecular weight of from about 0.5 to about 15 kDa. In one embodiment, the chitosan has a molecular weight of about 10 kDa. Suitable chitosans include oxidatively degraded chitosans prepared from commercially available chitosan.

The copolymer also includes a plurality of poly(ethylene oxide) oligomers. In one embodiment, poly(ethylene oxide) oligomers are grafted to the chitosan's backbone to provide a copolymer having pendant poly(ethylene oxide) oligomer side chains.

Suitable poly(ethylene oxide) oligomers include poly(ethylene oxides) (PEO or PEG) and poly(ethylene oxide) copolymers such as block copolymers that include poly(ethylene oxide) and poly(propylene oxide) (e.g., PEO-PPO and PEO-PPO-PEO). In one embodiment, the poly(ethylene oxide) oligomer is a poly(ethylene oxide). In certain embodiments, poly(ethylene oxide) oligomer has a molecular weight (weight average, Mw) of from about 0.3 to about 40 kDa. In others embodiments, the poly(ethylene oxide) oligomer has a molecular weight of from about 1.0 to about 10 kDa. In certain embodiments, the poly(ethylene oxide) oligomer has a molecular weight of about 2 kDa.

Representative chitosan-poly(ethylene oxide) oligomer copolymers include from about 2 to about 50 weight percent poly(ethylene oxide) oligomer. In one embodiment, the copolymer includes from about 5 to about 25 weight percent poly(ethylene oxide) oligomer.

Representative chitosan-poly(ethylene oxide) oligomer graft copolymers have a degree of poly(ethylene oxide) oligomer substitution of from about 0.01 to about 0.5. In certain embodiments, the graft copolymers have a degree of poly(ethylene oxide) oligomer substitution from about 0.01 to about 0.2. As used herein, the term “degree of substitution” or “DS” refers to the fraction of glucosamine repeating units in the chitosan that are substituted with a poly(ethylene oxide) oligomer. For DS=1.0, 100% of the glucosamine units are substituted with the poly(ethylene oxide) oligomer.

In this embodiment, the nanoparticle can be prepared by coating the core with the copolymer. In a representative method, the nanoparticle is formed by co-precipitation of iron oxide and the copolymer.

The method of the invention is effective to label and therefore detect select biological particles circulating in the vasculature of a living organism. In one embodiment, contacting the biological particle with a fusion protein includes administering the fusion protein to a subject to be diagnosed or treated. Suitable administration includes intravenous administration methods. Like the fusion protein, the nanoparticle including the binding partner can be administered intravenously. The fusion protein and/or the nanoparticle can be administered as a composition that includes a carrier suitable for administration to a human subject. Suitable carriers include those suitable for intravenous injection (e.g., saline or dextrose).

In other aspects, the invention provides methods for labeling and treating cancer cells. In one embodiment, the method for labeling cancer cells includes:

(a) contacting a cancer cell with a fusion protein having a biotin-binding region and a targeting region having an affinity toward the cancer cell to provide a fusion protein-labeled cancer cell; and

(b) contacting the fusion protein-labeled cancer cell with a biotinylated nanoparticle to provide a nanoparticle-labeled biological particle.

The invention also provides a method for treating cancer by delivering a chemotherapeutic (e.g., cytotoxic) agent to a cancer cell. In one embodiment, the method for treating cancer cells includes contacting a fusion protein-labeled cancer cell having an affinity to a binding partner with a nanoparticle comprising the binding partner and a chemotherapeutic agent.

The following is a description of the preparation and characteristics of a representative nanoparticle system and its labeling effectiveness in two model cancer cells (Ramos and Jurkat cells) using fusion proteins (anti-CD20 and anti-TAG-72 CC49 FPs). In these representative embodiments, a cell-targeting recombinant fusion protein composed of a single-chain antibody and streptavidin was utilized to specifically pre-label a targeted cell, followed by application of a biotinylated nanoparticle that binds to the streptavidin of the fusion protein labeled-target cell.

Nanoparticle synthesis and characterization. The base nanoparticles were produced by iron chloride co-precipitation in the presence of a biocompatible chitosan-poly(ethylene glycol) (PEG) copolymer, yielding chitosan-PEG-coated iron oxide nanoparticles (NP) with a mean core diameter of 7 nm, as described in U.S. patent application Ser. No. 12/384,923, filed Apr. 9, 2009 (US 2010/0260686), expressly incorporated herein by reference in its entirety. The chitosan-PEG copolymer provides a protective shell around the iron oxide nanoparticle core for enhanced nanoparticle biostability. The shell also provides functional groups (i.e., amine groups) for further conjugation of biomolecules. Addition of the PEG in the copolymer also limits the nanoparticle's overall cationic charge, reducing nonspecific interactions of the material with the cells. The free amino groups on the nanoparticle coat were modified with the Alexa Fluor 647 (AF647) fluorophore and biotin targeting agent using NHS chemistry, yielding the targeting, biotinylated nanoparticle formulation (NPB-AF647). The NP modified with the AF647 only (i.e., without biotin modification) served as control (NP-AF647). Because the biotin was coupled to the chitosan, aside the PEG co-polymer, a (PEO)₄ linker group was used between the biotin and chitosan to reduce the possibility of steric hindrance during biotin-streptavidin binding.

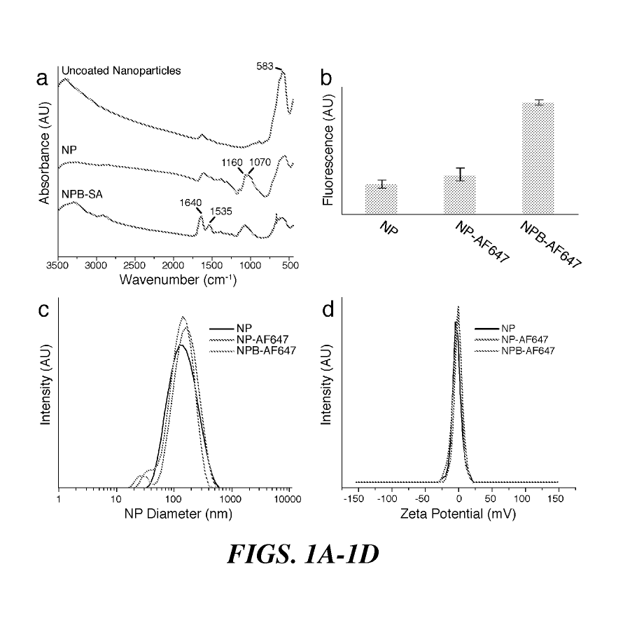

Successful nanoparticle coating and biotin functionalization was confirmed by infrared (FTIR) spectroscopy (see FIG. 1A). The absorption bands of the chitosan-PEG were observed on the spectrum of chitosan-PEG coated nanoparticles (NPs) at 1160 (asymmetric stretching of C—O—C) and 1075-1033 cm (C—O stretching), and all nanoparticle samples showed the Fe—O and O—H absorption bands at 583 and 3400 cm⁻¹. To verify biotin-binding activity of biotinylated nanoparticles (NPBs), the NPBs were mixed with streptavidin (SA) to provide streptavidin labeled with biotinylated nanoparticles (NPB-SA), which were rinsed and was analyzed by FTIR. Amide I (1640 cm⁻¹) and amide II (1535 cm⁻¹) peaks were observed indicating positive SA binding (see FIG. 1A). The functional activity of the biotin was further confirmed by the FluoReporter Biotin assay (see FIG. 1B), an assay that identifies active biotin labels by the displacement of a quencher dye from biotin binding sites on a fluorescent avidin probe. In the assay, the nanoparticles with no biotin (i.e., NP and NP-AF647) served as controls and did not significantly interact with the quenched probe, while NPB-AF647 showed significant biotin interaction, yielding an increase in fluorescence, indicating specific binding to avidin (see FIG. 1B).

Physical analysis of the NP and NPB formulations showed that little size change occurred during the functionalization of the nanoparticles with fluorophore and biotin (see FIG. 1C). The average hydrodynamic size of NP-AF647 and NPB-AF647 increased by <20 nm over NP. Importantly, the addition of biotin to the nanoparticle surface resulted in substantially no change in the zeta potential of the nanoparticle (see FIG. 1D).

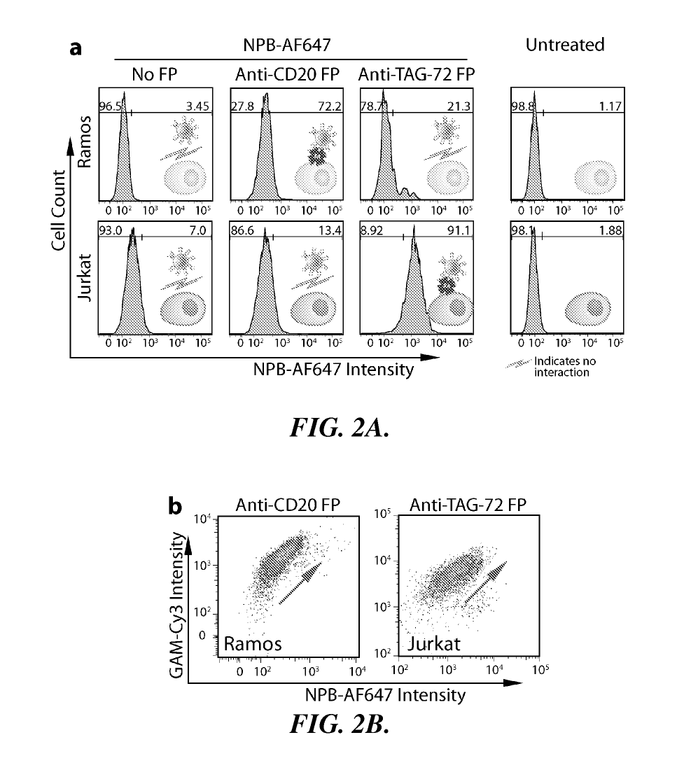

Selective uptake of biotinylated nanoparticles by target cells. Binding specificity of NPB-AF647 for cells pretreated with FPs was evaluated using CD20⁺/TAG72⁻ Ramos and CD20⁻/TAG72⁺ Jurkat cell lines (American Type Culture Collection, Manassas, Va.). Each cell line was treated with either anti-CD20 or anti-TAG-72 CC49 FPs, which are routinely used as the targeting agents for labeling non-Hodgekin's lymphoma and adenocarcinoma, respectively. Cells treated with the FPs or receiving no FP pretreatment were then exposed to NPB-AF647 and evaluated by flow cytometry (see FIG. 2A). Cells that received neither FP nor NPB-AF647 treatment were also evaluated (controls) (See FIG. 2, fourth column “Untreated”).

Ramos cells (CD20⁺/TAG72⁻) showed positive NPB-AF647 binding (72.2%) when pretreated with the anti-CD20 FP, but limited NPB-AF647 labeling (21.3%) when pretreated with the anti-TAG-72 FP, and minimal NPB-AF647 labeling (3.45%) when receiving no FP pretreatment. The difference in labeling between these two controls may be due to the non-specific binding of the anti-TAG-72 FP to the Ramos cells. Jurkat cells (CD20⁻/TAG72⁺) showed positive labeling (91.1%) when pretreated with anti-TAG-72 CC49 FP, but limited NPB-AF647 binding (13.4%) when pretreated with the anti-CD20 FP, and minimal NPB-AF647 labeling (7%) when receiving no FP pretreatment. Control nanoparticles (NP-AF647), with no biotin functionalization, showed no specific cell binding regardless of the cells' FP pretreatment. Because anti-CD20 FP binds to Ramos, but not Jurkat cells, and anti-TAG-72 FP binds to Jurkat, but not Ramos cells, the results indicate that NPB-AF647 preferentially binds to cells that bear their complementary FPs. The result was further supported by the fact that NPB-AF647 bound minimally to both cell lines that received no FP pretreatment (see FIG. 2A, first column “No FP”). The results also confirmed the binding activity of the biotin conjugated on the nanoparticle in addition to the confirmation by the FTIR and fluorescence analyses (see FIGS. 1A and 1B, respectively).

To further confirm that NPB-AF647 was bound to cells labeled with the murine-derived FPs, Ramos and Jurkat cells were pretreated with anti-CD20 and anti-TAG-72 CC49 FPs, respectively, and then exposed to NPB-AF647. The cells were then co-stained with a fluorescent goat anti-mouse (GAM-Cy3) secondary antibody, which binds to the SA of the FPs, and analyzed by flow cytometry (see FIG. 2B). As observed in both cell lines, higher GAM-Cy3 staining correlated with increased nanoparticle binding (denoted by the arrows), confirming specific targeting of NPB-AF647 to cells with positive FP expression.

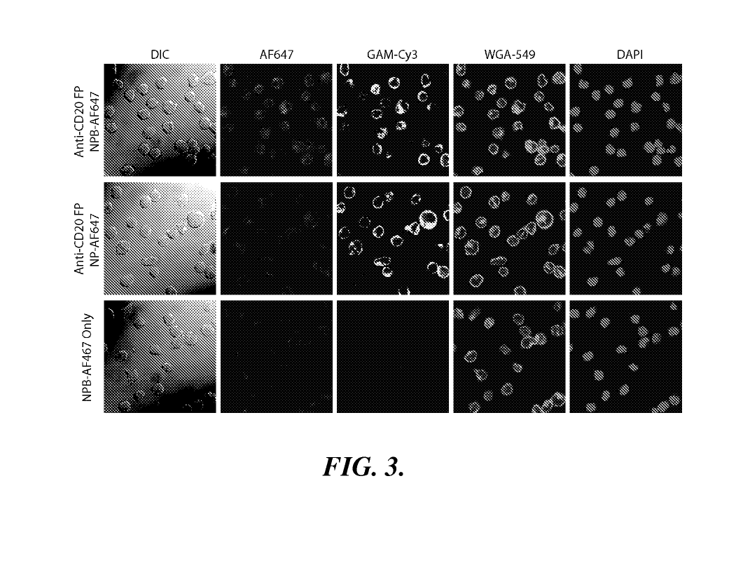

Endocytosis of Nanoparticles by Target Cells. Selective binding of the NPB-AF647 to Ramos cells pre-treated with anti-CD20 FP was further visualized by confocal and differential interference (DIC) microscopy. Confocal imaging showed nanoparticle-cell co-localization, while DIC imaging corroborated cell location (see FIG. 3). Ramos cells pre-treated with anti-CD20 FP (target cells) or receiving no pretreatment (control cells) were exposed to either NPB-AF647 (targeting particles) or NP-AF647 (non-targeting particle controls). Fluorescence imaging of the nanoparticles (AF647), FPs (GAM-Cy3), cell membranes (WGA-594), and nuclei (DAPI) was performed. Yellow fluorescence observed on Ramos cells pretreated with anti-CD20 FP (rows 1 and 2) and the co-localization of yellow and green fluorescence indicate the positive FP labeling on the cell surfaces. Strong red fluorescence was observed on the Ramos cells pretreated with anti-CD20 FP (row 1), but not with cells that did not receive anti-CD20 FP pretreatment (row 3), after both were exposed to NPB-AF647, indicating that NPB-AF647 bound specifically to the target cells. Substantially no red fluorescence was observed on Ramos cells exposed to NP-AF647 controls (row 2), as expected, even though the cells were pre-treated with anti-CD20 FP. Notably, nanoparticle fluorescence (AF647) was co-localized with GAM-Cy3, indicating that the targeted cell binding by NPB-AF647 was via the SA of the FPs bound to the cell (row 1).

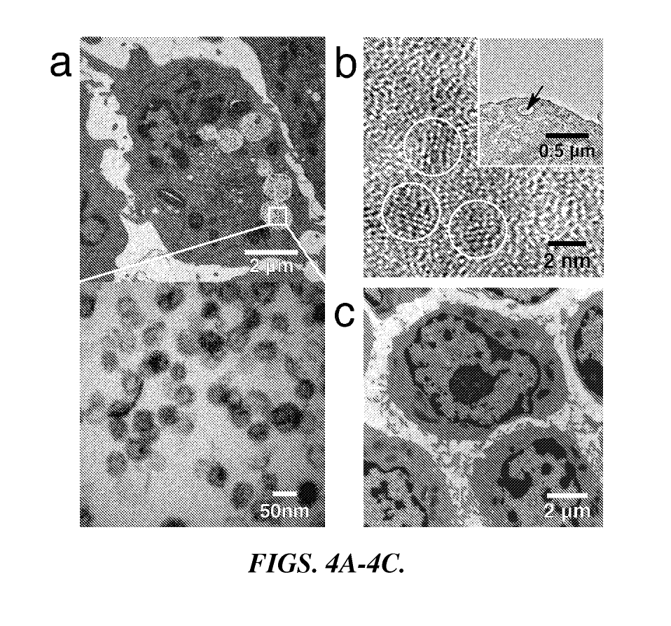

Transmission electron microscopy (TEM) was used to examine the localization of the nanoparticles bound to target cells. TEM micrographs showed that a large population of nanoparticles were internalized by the Ramos cells and localized in the endosomes having diameters on the order of 1 μm (see FIG. 4A, top image). TEM imaging at higher magnification further revealed that the nanoparticles were well dispersed in the endosomes and exhibited a uniform size distribution (see FIG. 4A, bottom image). At even higher magnification (see FIG. 4B), TEM analysis showed that the nanoparticles in the endosome (see FIG. 4B inset) had characteristic lattice fringe patterns of the iron oxide crystals (circled in FIG. 4B). Further confirmation of nanoparticle internalization by cells was made by energy-dispersive X-ray spectroscopy (EDX), where a cluster of nanoparticles inside a cellular endosome was probed, showing characteristic iron peaks. Similar probing elsewhere in the cell showed the presence of minimal or no iron (data not shown). Electron micrographs of Ramos cells treated with NPB-AF647, but with no FP pretreatment, showed no evidence of nanoparticle internalization in cells (see FIG. 4C). This visually validated the flow cytometry results shown above.

The underlying mechanism of the particle internalization by Ramos cells that bear CD20, a non-internalizing epitope target, may be attributed to the physicochemical profile of the nanoparticle (coated with a chitosan-PEG graft co-polymer).

The following examples are provided for the purpose of illustrating, not limiting, the invention.

### EXAMPLES

### Example 1

### Nanoparticle Preparation and Characterization

In this example, the preparation and characterization of a representative nanoparticle useful in the method of the invention is described.

Nanoparticle preparation. The iron oxide nanoparticles were synthesized and coated with the chitosan-PEG polymer by co-precipitating Fe(II) and Fe(III) with the addition of sodium hydroxide in the presence of the polymer, as described in U.S. patent application Ser. No. 12/384,923, filed Apr. 9, 2009 (US 2010/0260686), expressly incorporated herein by reference in its entirety. The chitosan-PEG coated nanoparticles (NPs) were then transferred into a 0.1M sodium bicarbonate pH 8.5 buffer by gel permeation chromatography (GPC) using Sephacryl S200 medium (GE Healthcare, Piscataway, N.J.). The NPs were then fluorescently labeled with Alexa Fluor 647 (AF647) by dissolving 1 mg AF647 succinimidyl ester (Invitrogen, Carlsbad, Calif.) in 0.15 mL dimethylsulfoxide (DMSO) and mixing with the NPs (3.5 mg Fe; about 2 mg Fe/mL) at room temperature for 2 hrs. Free AF647 was removed by GPC purification using phosphate buffered saline (PBS) pH 7.4 (Sephacryl S200) to form control nanoparticles (NP-AF647).

Biotinylated NP-AF647 (NPB-AF647) was prepared by dissolving 2 mg EZ-link NHS-PEG₄-Biotin (Thermo Fisher Scientific, Rockford, Ill.) in 100 μL 0.1M sodium bicarbonate pH 8.5 and adding to 1.75 mg Fe of the unpurified NP/AF647 mixture. The mixture was allowed to stand at room temperature for 2 hrs, then purified using PBS pH 7.4 by GPC (Sephacryl S200), yielding the NPB-AF647.

Nanoparticle characterization. For FTIR analysis, nanoparticle samples were milled with potassium bromide (KBr) and pressed into pellets. FTIR spectra were acquired with a Nicolet 5-DXB spectrometer with a resolution of 4 cm⁻¹. For NPB-AF647, the nanoparticles were mixed with SA (0.1 mg/mL; Invitrogen) for 30 min, washed of unbound SA on a rare earth magnet 3×, and analyzed by FTIR. For FluoReporter Biotin Assay analysis, nanoparticles were dissolved in 1M HCl for 2 hrs at 35° C. before being neutralized. Dissolved nanoparticle samples had a final Fe concentration of 50 μg/mL. All samples were mixed with the Biotective Green reagent for 10 min and fluorescence was measured at 495/519 nm (ex/em; SpectraMax microplate reader). Nanoparticle sizes and zeta-potentials were determined by light scattering using a Zetasizer ZS (Malvern Instruments, Southborough, Mass.). All samples were analyzed in PBS pH 7.4 and sizing was completed using z-average data acquisition.

### Example 2

### Fusion Protein Production

In this example, the production of representative fusion proteins useful in the method of the invention is described.

The heavy-chain (V_{H}) and light-chain (VL) genes were cloned from the 1F5 (anti-CD20) and CC49 (anti-TAG-72) hybridomas. The single-chain antibody-SA genes were engineered by fusing the scFv gene to the full-length genomic SA of Streptomyces avidinii as described in J Immunol, 1999, 162, 6589-6595. The FPs were expressed from an IPTG-inducible lac promoter. An E coli XL1 Blue (Stratagene, La Jolla, Calif.) transformant of the antibody-SA construct was grown in shaker flasks for qualitative expression of the FPs and subsequently in a 4 L fermentor (BioFlo 3000; New Brunswick Scientific, Edison, N.J.) using methods described in Cancer Res, 2000, 60, 6663-6669. Cultures were induced with 0.1 mM IPTG and cells harvested after 44 hrs. The cell paste was washed 3x with PBS and the lysate was purified by iminobiotin column chromatography. The eluted FPs were treated with 20% DMSO for 2 hrs to reduce aggregates, extensively dialyzed in PBS, filter-sterilized, formulated in 5% sorbitol, and stored at −80° C.

### Example 3

### Representative Cell Labeling and Characterization

In this example, a representative method for labeling cells in accordance with the method of the invention is described.

Cell Pre-Labeling

Ramos and Jurkat cells were cultured in RPMI media modified with 10% FBS and 1% penicillin and streptomycin (Invitrogen) at 37° C. under 5% carbon dioxide. To test nanoparticle affinity and specificity, 10⁶ cells/sample were washed into 100 μL PBS and incubated with 10 μg of either anti-CD20 [1F5-(sFv)₄-streptavidin] or anti-TAG-72 [CC49-(scFv)₄-streptavidin] FP for 30 min at 4° C. and washed 3× with PBS by centrifugation. Cell samples were then incubated with either NP-AF647 or NPB-AF647 at 0.1 mg Fe/mL in PBS for 1 hr at 4° C. Cells were washed 3× with PBS, and incubated with 10 μg goat-antimouse-Cy3 (GAM-Cy3) for 30 min at 4° C. Samples were then analyzed by flow cytometry (FACS Canto, BD Biosciences, San Jose, Calif.). 20,000 cells were analyzed from each sample. For each condition, n=3 samples were analyzed in each experiment, and each experiment was at least repeated twice to confirm the result.

Confocal microscopy. Three million Ramos cells, incubated with either NPB-AF647 (with and without anti-CD20 FP pretreatment) or NP-AF647 (with anti-CD20 FP pretreatment), were washed with PBS 3×, stained with GAM-Cy3, washed again with PBS 3×, and fixed in a 4% formaldehyde/PBS solution (methanol free, Polysciences Inc., Warrington, Pa.) for 30 min. The fixative was then removed and cells washed with PBS 3×. The cell membranes were then stained with wheat germ agglutinin-AlexaFluor 594 (WGA-AF594; Invitrogen) according the manufacturer's instructions, and incubated with DAPI-containing Prolong Gold antifade solution (Invitrogen) for cellular nuclei staining and fluorescence preservation. Cell samples were then dried on cover slips and imaged using a Zeiss LSM 510 Meta confocal fluorescence microscope (Peabody, Mass.).

Transmission electron microscopy. One million Ramos cells, pretreated with the FP and incubated with NPB-AF647, were washed 3× with PBS and immersed in ice cold Karnovsky's fixative for 24 hrs. Following fixation, the cells were pelletized, sectioned, and stained with osmium tetroxide, lead citrate, and uranyl acetate for TEM-contrast enhancement. Cell samples were mounted onto carbon-coated copper grids. Micrographs were taken with a Philips CM100 operating at 100 kV (low magnification) and a Tecnai G2 F20 TEM operating at 200 kV (high magnification). EDX spectra were obtained on the Tecnai TEM equipped with an EDAX detector.

While illustrative embodiments have been illustrated and described, it will be appreciated that various changes can be made therein without departing from the spirit and scope of the invention.

1. A method for labeling a biological particle, comprising:
(a) contacting a biological particle with a fusion protein having a binding region having an affinity to a binding partner and a targeting region having an affinity toward the biological particle to provide a fusion protein-labeled biological particle; and
(b) contacting the fusion protein-labeled biological particle with a nanoparticle comprising the binding partner to provide a nanoparticle-labeled biological particle.

2. The method of claim 1, wherein contacting the biological particle with a fusion protein comprises administering the fusion protein to a subject to be diagnosed or treated.

3. The method of claim 2, wherein administering the fusion protein comprises intravenous administration.

4. The method of claim 1, wherein the biological particle is a particle circulating in the vasculature of a living organism.

5. The method of claim 1, wherein the biological particle is a cell, a bacterium, a virus or viral particle, or a biomarker.

6. The method of claim 5, wherein the cell is a stem cell, a blood cell, or a tissue cell.

7. The method of claim 6, wherein the stem cell is an embryonic stem cell or adult stem cell.

8-12. (canceled)

13. The method of claim 5, wherein the fusion protein binding region comprises a biotin binding region.

14. The method of claim 5, wherein the fusion protein binding region comprises an avidin, a streptavidin, a neutravidin, or functional fragments thereof.

15. The method of claim 5, wherein the fusion protein targeting region comprises a single chain antibody or functional fragment thereof.

16. (canceled)

17. The method of claim 1, wherein the nanoparticle further comprises an imaging agent.

18. (canceled)

19. The method of claim 1, wherein the nanoparticle further comprises a therapeutic agent.

20-21. (canceled)

22. The method of claim 20, wherein the gene therapeutic agent is a DNA or an RNA.

23-26. (canceled)

27. The method of claim 1, wherein the nanoparticle further comprises an imaging agent and a therapeutic agent.

28. The method of claim 1, wherein the nanoparticle is a magnetic nanoparticle.

29-31. (canceled)

32. The method of claim 1, wherein the nanoparticle comprises:
(a) a core having a surface and comprising a core material; and
(b) a coating on the surface of the core, the coating comprising a copolymer comprising a chitosan and a poly(ethylene oxide) oligomer.

33. The method of claim 32, wherein the core material is a magnetic material.

34-35. (canceled)

36. A method for labeling a cancer cell, comprising:
(a) contacting a cancer cell with a fusion protein having a biotin-binding region and a targeting region having an affinity toward the cancer cell to provide a fusion protein-labeled cancer cell; and
(b) contacting the fusion protein-labeled cancer cell with a biotinylated nanoparticle to provide a nanoparticle-labeled biological particle.

37-38. (canceled)

39. A method for labeling a biological particle, comprising contacting a fusion protein-labeled biological particle having an affinity to a binding partner with a nanoparticle comprising the binding partner to provide a nanoparticle-labeled biological particle.

40. A method for treating cancer by delivering a chemotherapeutic agent to a cancer cell, comprising contacting a fusion protein-labeled cancer cell having an affinity to a binding partner with a nanoparticle comprising the binding partner and a chemotherapeutic agent.

41-42. (canceled)
